(19) United States
(12) Plant Patent Application Publication
(10) Pub. No.: US 2003/0226186 P1
(43) Pub. Date: Dec. 4, 2003

(54) BEGONIA PLANT NAMED 'BBSIRIUS'

(75) Inventor: Bert Koeze, Nieuw Vennep (NL)

Correspondence Address: Russel D. Orkin, 700 Koppers Building, 436 Seventh Avenue, Pittsburgh, PA, 15219-1818, US

(73) Assignee: Begonia Breeders Association B.V.

(21) Appl. No.: 10/157,365

(22) Filed: May 28, 2002

(51) Int. Cl. A01H 5/00

(52) U.S. Cl. PLT/348

(57) ABSTRACT

A new and distinct begonia plant with red colored flowers.

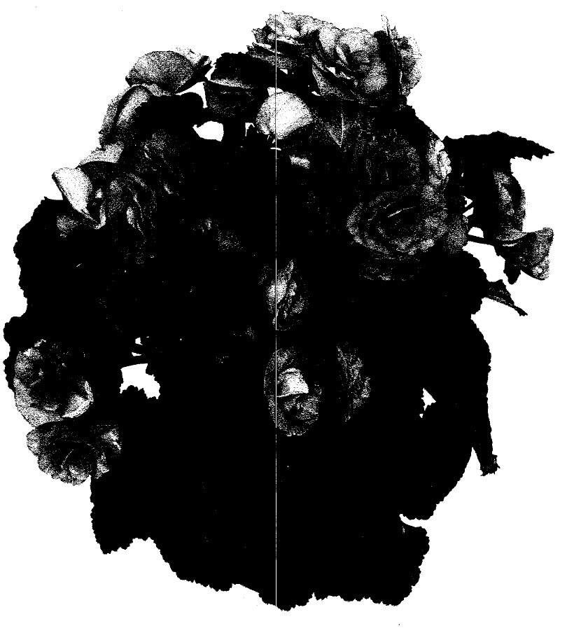

### VARIETAL DENOMINATION

‘98238-3’

### BOTANICAL CLASSIFICATION

Begonia Elatior (Begonia)

### BACKGROUND OF THE INVENTION

The present invention comprises a new and distinct cultivar of Begonia elatior known by the varietal name ‘98238-3’. The new variety was discovered in Aalsmeer, Netherlands, in 1998. The new variety is a product of a planned breeding program and is a cross of an unnamed, unpatented variety of Begonia tuberosa (female) and an unnamed, unpatented variety of Begonia socotrana (male). The breeding program is directed to establishing various phenotype characteristics. The new variety differs in its flower color and its double flowers as compared to its parents. The new variety was first asexually reproduced by cuttings in Aalsmeer, Netherlands. The new variety has been trial and field tested and has been found to retain its distinctive characteristics and remain true to type through successive propagations.

The new variety, when grown in a glass greenhouse has a response time for rooting from a terminal cutting of 14-18 days.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions by cuttings in a controlled greenhouse environment. Color references are primarily to the R.H.S. Colour Chart of The Royal Horticultural Society of London.

### PLANT

Form: Upright.

Growth: Moderate.

Time to initiate roots: About 14-18 days.

Time to develop roots: 30 days.

Rooting habit: Fine, fibrous, well-branched.

Height: 30 cm from the media surface to the top of the leaf plane.

Width: 40 cm.

Lateral branches: 4-5 in number.

Leaves:

Arrangement.—Alternate.

Shape.—Rounded.

Size.—4-6 cm long; 5-6 cm wide.

Quantity.—Numerous.

Petiole.—4-5 cm long; 5 mm diameter; green in color.

Stipules.—1 cm.

Margin.—Irregular; serrated.

Texture.—Smooth; shiny and leathery.

Color.—146D.

Venation.—Palmate.

### FLOWERS

Habit: Continuous flowering.

Shape: Rounded.

Aspect: Flat to cupped.

Size: 5-6 cm in diameter; 1-1.5 cm deep.

Fragrance: None.

Flower buds:

Size.—2-3 cm long; 2.5 cm wide.

Color.—50B.

Rate of flower opening.—3 days.

Petals:

Arrangement.—Double.

Shape.—Oval.

Margin.—Entire with irregularities.

Quantity.—15-20 per flower.

Size.—2.5 cm long; 4 cm wide.

Texture.—Smooth.

Color.—57B, both surfaces.

Peduncles:

Angle.—Mostly erect.

Strength.—Strong.

Length.—3-4 cm.

Color.—47A.

Pedicels:

Angle.—About 30 to 45° from vertical.

Strength.—Strong.

Length.—2.0 cm.

Color.—50B.

### REPRODUCTIVE ORGANS

Stamens: None observed.

Pistils: None observed.

Disease resistance: Mildew tolerant.

Seed production: Seed production has not been observed as reproductive organs are not formed.

I claim:

1. A new and distinct variety of begonia plant substantially as shown and described.
